SEQUENCE LISTING (2 sequences)

SEQ ID NO: 1 - protein, 381 aa, Bacillus sphaericus
MAKQLEKSSKIGNEDVFQKIANHEQIVFCNDPVSGLQAIIAIHDTTLGPALGGTRMYPYKNVDEALEDVLRLSEGMTYKCAAADIDFGGGKAVIIGDPEKDKSPALFRAFGQFVESLNGRFYTGTDMGTTMDDFVHAQKETNFINGIPEQYGGSGDSSIPTAQGVIYALKATNQYLFGSDSLSGKTYAIQGLGKVGYKVAEQLLKAGADLFVTDIHENVLNSIKQKSEELGGSVTIVKSDDIYSVQADIFVPCAMGGIINDKTIPKLKVKAVVGSANNQLKDLRHANVLNEKGILYAPDYIVNAGGLIQVADELYGPNKERVLLKTKEIYRSLLEIFNQAALDCITTVEAANRKCQKTIEGQQTRNSFFSRGRRPKWNIKE

SEQ ID NO: 2 - DNA, 1146 bp, Bacillus sphaericus
atggcaaaacagcttgaaaagtcatcaaaaattggtaatgaggacgtttttcaaaaaatagcgaatcacgagcagattgtgttctgtaatgatccggtatccggcctgcaagctatcattgctatccacgatacaaccctaggccccgctttaggtggaactcgcatgtatccctataaaaatgtggatgaagctctggaagatgtgcttcgcctgtcagaaggaatgacgtataaatgcgcagccgccgatatcgatttcggcggcgggaaggcggtcattatcggagatccagaaaaggataaatctccggcattgttccgtgcatttggtcaatttgtggaatcactgaatggacgattttacacaggtactgacatggggaccacgatggatgattttgtccatgcacagaaagagacgaatttcattaacggaattcctgagcagtatggtggaagcggcgactcgtcgattccgaccgcccagggagtcatttatgcactgaaggctacaaaccagtatttatttggaagcgatagcctttcaggtaaaacatatgctattcaagggctgggaaaagtagggtataaagtagcggaacagctcttaaaagccggcgccgatttatttgtaacggatatacatgaaaatgtcctcaattccattaagcaaaaatcagaagagcttggcggttcagtgaccattgtaaaaagtgacgatatttacagcgtacaagcggatatatttgttccgtgtgcgatgggtggtattatcaatgataaaaccattcctaagttaaaggtgaaggctgttgtgggatcagccaataaccagctcaaagacctccgccatgcaaatgtactaaacgaaaagggaattctatatgcacccgattatatcgtcaatgccggcggcttgatccaggttgctgacgaactttatgggccgaataaagagcgggtcttgctcaaaacgaaagaaatttaccgttctctgcttgaaatttttaatcaggcagcccttgactgcatcacaacagtggaggccgcaaataggaagtgtcaaaagacgattgagggccagcaaacccgtaatagtttcttttctaggggacgcaggccgaagtggaacataaaagagtaa

(19) United States
(12) Patent Application Publication
(10) Pub. No.: US 2016/0153015 A1
(43) Pub. Date: Jun. 2, 2016

(54) SYNTHESIS METHOD FOR L-HETEROCYCLIC AMINO ACID AND PHARMACEUTICAL COMPOSITION HAVING THEREOF

(71) Applicants: ASYMCHEM LABORATORIES (TIANJIN) CO., LTD, Tianjin (CN); ASYMCHEM LIFE SCIENCE (TIANJIN) CO., LTD, Tianjin (CN); TIANJIN ASYMCHEM PHARMACEUTICAL CO., LTD, Tianjin (CN); ASYMCHEM LABORATORIES (FUXIN) CO., LTD, Liaoning (CN); JILIN ASYMCHEM LABORATORIES CO., LTD, Jilin (CN)

(72) Inventors: Hao HONG, TEDA Tianjin (CN); Changsheng ZHENG, TEDA Tianjin (CN); Lina GUO, TEDA Tianjin (CN)

(21) Appl. No.: 14/782,586

(22) PCT Filed: Jun. 27, 2013
(86) PCT No.: PCT/CN2013/078154

§ 371 Date: Oct. 5, 2015

(51) Int. Cl. C12P 17/12; C12P 17/00; A61K 31/4409; A61K 38/44; A61K 31/4406; A61K 31/415; A61K 31/422; C12P 17/10; A61K 31/4402

(52) U.S. Cl. CPC C12P 17/12; C12P 17/10; C12P 17/00; A61K 31/4409; A61K 31/4402; A61K 31/4406; A61K 31/415; A61K 31/422; A61K 38/44; C12Y 104/0102; C12Y 102/01002; A61K 48/00

(57) ABSTRACT

A synthesis method for an L-heterocyclic amino acid and a pharmaceutical composition having the said amino acid are provided in the present disclosure. The synthesis method comprises: step A: preparing a heterocyclic keto acid, wherein the heterocycle in the heterocyclic keto acid is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, and the keto acid group in the heterocyclic keto acid has a structural formula of

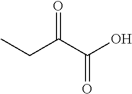

and is located on any one of the carbon positions of the heterocycle, and step B: mixing the heterocyclic keto acid with ammonium formate, a phenylalanine dehydrogenase, a formate dehydrogenase and a coenzyme NAD⁺, and carrying out a reductive amination reaction to generate L-heterocyclic amino acid, wherein the amino acid sequence of the phenylalanine dehydrogenase is SEQ ID No. 1.

### TECHNICAL FIELD

The present disclosure relates to the field of medicine synthesis, and particularly to a synthesis method for L-heterocyclic amino acid and a pharmaceutical composition having the said amino acid.

### BACKGROUND

At present, non-natural chiral heterocyclic amino acids are mainly synthesized chemically, including methods such as single-configuration conversation implemented on a certain key intermediate by asymmetric catalytic hydrogenation of a noble metal, resolution of a racemate by using a chiral reagent, asymmetric synthesis using a chiral auxiliary, rational synthesis using a chiral raw material, and the like. However, these methods have the following disadvantages:
(1) implementation of single-configuration conversation for a certain key intermediate by an asymmetric catalytic hydrogenation of a noble metal has the following disadvantages: the noble metal asymmetric catalyst is expensive, a large amount of organic solvent is needed in the reaction, there are heavy metal residues in a product and there may be excessive reduction by-products in the product; in addition, binding of the noble metal and a ligand is usually interfered by a heterocycle contained in a synthesis raw material, which results in low catalytic efficiency;
(2) an isomer required in a racemate is obtained by applying a traditional chiral resolving method, which may cause waste of the other half of raw materials;
(3) asymmetric synthesis using a chiral auxiliary or a chiral raw material involves expensive chiral raw materials, long synthesis route and a large amount of organic solvent, in addition, products obtained in synthesis of some heterocyclic amino acids are low in optical purity, or the products can be hardly separated from impurities.

It is also reported in some literatures in the prior art that some simple alkyl keto acids are catalyzed by specific enzymes to be converted into corresponding amino acids through biosynthesis. However, in the prior art, because the heterocyclic amino acids have relatively special properties, there are no proper enzymes and corresponding reaction conditions can be used in biotransformation for synthesizing chiral heterocyclic amino acids.

### SUMMARY

The present disclosure aims at providing a synthesis method for L-heterocyclic amino acid and a pharmaceutical composition having the said amino acid to obtain an L-heterocyclic amino acid with relatively high optical purity.

To realize the purpose above, a synthesis method for L-heterocyclic amino acid is provided according to an aspect of the present disclosure, the synthesis method comprises: Step A: preparing a heterocyclic keto acid, wherein the heterocycle in the heterocyclic keto acid is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, and wherein the keto acid group in the heterocyclic keto acid has a structural formula of:

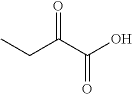

and is located on any one of the carbon positions of the heterocycle, and step B: mixing the heterocyclic keto acid with ammonium formate, a phenylalanine dehydrogenase, a formate dehydrogenase and a coenzyme Nicotinamide Addenine Dinucleotide (NAD⁺), and carrying out a reductive amination reaction to generate the L-heterocyclic amino acid, wherein the amino acid sequence of the phenylalanine dehydrogenase is SEQ ID No. 1.

Further, a gene sequence coding the phenylalanine dehydrogenase is SEQ ID No. 2.

Further, an expression process of the phenylalanine dehydrogenase comprises: inserting a Deoxyribonucleic Acid (DNA) fragment containing the gene sequence into a vector to obtain a gene recombinant plasmid; transferring the gene recombinant plasmid to a host strain and culturing the host strain on a culture medium, and inducing production of the phenylalanine dehydrogenase by an inducer; breaking the host strain with ultrasonic waves, and then carrying out centrifugal separation to obtain a crude enzyme mixed solution which contains the phenylalanine dehydrogenase and the formate dehydrogenase.

Further, in the crude enzyme mixed solution, the specific enzyme activity of the phenylalanine dehydrogenase is 40 U/ml to 60 U/ml, and the specific enzyme activity of the formate dehydrogenase is 20 U/ml to 30 U/ml.

Further, the Step B comprises: adding the heterocyclic keto acid and ammonium formate to an aqueous solution, regulating the pH value to 8.2 to 8.5, adding the crude enzyme mixed solution and the coenzyme NAD⁺, and performing reaction at 30° C. to 40° C. until conversion of the raw materials is finished to obtain the L-heterocyclic amino acid.

Further, 2 ml to 10 ml of the crude enzyme mixed solution is added to each mole of the heterocyclic keto acid; 0.005 mole to 0.1 mole of the coenzyme NAD⁺ is added to each mole of the heterocyclic keto acid and 1.5 moles to 5 moles of ammonium formate is added to each mole of the heterocyclic keto acid.

Further, after the Step B, the synthesis method further comprises: adding concentrated hydrochloric acid to the system after the reaction, passing the system with the concentrated hydrochloric acid through diatomite to obtain a filtrate; regulating the pH value of the filtrate to 5.0 to 7.0, then passing the filtrate through a strong acid cation exchange resin to obtain a crude product; concentrating the crude product, adding an alcoholic solvent to wash the crude product and drying the washed crude product to obtain a purified L-heterocyclic amino acid.

Further, a method for preparing the heterocyclic keto acid comprises the following steps: subjecting a heterocyclic ketone with an acetic anhydride, a sodium acetate and an N-acetylglycine to reaction to obtain an intermediate product, wherein the heterocycle in the heterocyclic ketone is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, the structural formula of the ketone group in the heterocyclic ketone is —C═O and is located on any one of the carbon positions of the heterocyclic ketone; subjecting the intermediate product to a hydrolysis reaction in the presence of a Lewis base, and acidizing to obtain the heterocyclic keto acid.

Further, a method for preparing the heterocyclic keto acid comprises the following steps: subjecting a heterocyclic alkyl compound with a diethyl oxalate in the presence of an N-butyllithium or a potassium tert-butoxide to reaction to generate a heterocyclic keto ester, wherein the heterocycle in the heterocyclic alkyl compound is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, the alkyl in the heterocyclic alkyl compound is methyl and is located on any one of the carbon positions of the heterocyclic alkyl compound; subjecting the heterocyclic keto ester to a hydrolysis reaction in the presence of a Lewis base, and acidizing to obtain the heterocyclic keto acid.

A pharmaceutical composition is provided according to another aspect of the present disclosure, the pharmaceutical composition comprises an effective dose of a L-heterocyclic keto acid and a pharmaceutical vector, the L-heterocyclic keto acid is synthesized and obtained by the synthesis method according to any one of claims 1 to 9.

Applying the technical solution of the present disclosure, a specific phenylalanine dehydrogenase having the amino acid sequence of SEQ ID No. 1, the formate dehydrogenase and the coenzyme NAD⁺ are used together to enable a reductive amination reaction of a heterocyclic keto acid so as to generate an L-heterocyclic amino acid, a chiral center is formed through conversion catalyzed by the phenylalanine dehydrogenase and the coenzyme, the conversion rate of raw materials is as high as above 80% with high chiral selectivity, and it does not need to separate and purify an isomer from an obtained product, thus further simplifying a synthesis process of the L-heterocyclic amino acid; in addition, reaction conditions in the whole synthesis process are moderate, which is more applicable to mass industrial production of the L-heterocyclic amino acid.

### DETAILED DESCRIPTION OF THE EMBODIMENTS

It should be noted that, if there is no conflict, the embodiments in the application and the characteristics in the embodiments can be combined with each other. The present disclosure will be described in details below in combination with the embodiments.

A synthesis method for L-heterocyclic amino acid is provided in a typical embodiment of the present disclosure, the synthesis method comprises: Step A: preparing a heterocyclic keto acid, wherein the heterocycle in the heterocyclic keto acid is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, and wherein the keto acid group in the heterocyclic keto acid has a structural formula of:

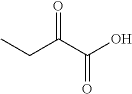

and is located on any one of the carbon positions of the heterocycle, and step B: mixing the heterocyclic keto acid with ammonium formate, a phenylalanine dehydrogenase, a formate dehydrogenase and a coenzyme NAD⁺, and carrying out a reductive amination reaction to generate the L-heterocyclic amino acid, wherein the amino acid sequence of the phenylalanine dehydrogenase is SEQ ID No. 1.

The synthesis method applies a specific phenylalanine dehydrogenase having the amino acid sequence of SEQ ID No. 1, the formate dehydrogenase and the coenzyme NAD+ together to enable a reductive amination reaction of a heterocyclic keto acid so as to generate an L-heterocyclic amino acid, a chiral center is formed through conversion catalyzed by the phenylalanine dehydrogenase and the coenzyme, the conversion rate of raw materials is as high as above 80% with high chiral selectivity, and it does not need to separate and purify an isomer from an obtained product, thus further simplifying a synthesis process of the L-heterocyclic amino acid, in addition, reaction conditions in the whole synthesis process are moderate, which is more applicable to mass industrial production of the L-heterocyclic amino acid.

The heterocycle of the present disclosure is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, wherein the five-membered heterocycle and the alkyl-substituted five-membered heterocycle include but are not limited to pyrrole, imidazole, triazole, furan, pyrazole, thiophene and corresponding chemically acceptable alkyl-substituted heterocycles thereof; the six-membered heterocycle and the alkyl-substituted six-membered heterocycle include, but are not limited to pyridine, pyrimidine, pyrazine, pyridazine and corresponding chemically acceptable alkyl-substituted heterocycles thereof; the seven-membered heterocycle and the alkyl-substituted seven-membered heterocycle include, but are not limited to indole, quinoline, pteridine, acridine or corresponding chemically acceptable alkyl-substituted heterocycles thereof, and wherein the alkyl is selected from any one of methyl, ethyl, propyl, and butyl, preferably methyl. In the synthesis method, a gene sequence coding the phenylalanine dehydrogenase is SEQ ID No. 2.

The phenylalanine dehydrogenase coded by the gene sequence has higher selectivity and catalytic conversion rate for catalyzing the synthesis of the L-heterocyclic amino acid from the heterocyclic keto acid and ammonium formate.

In a preferred embodiment of the present disclosure, an expression process of the phenylalanine dehydrogenase comprises: inserting a DNA fragment containing the gene sequence into a vector to obtain a gene recombinant plasmid; transferring the gene recombinant plasmid to a host strain and culturing the host strain on a culture medium, and inducing production of the phenylalanine dehydrogenase by an inducer; breaking the host strain with ultrasonic waves, and then carrying out centrifugal separation to obtain a crude enzyme mixed solution which contains the phenylalanine dehydrogenase and the formate dehydrogenase.

The activity and content of the phenylalanine dehydrogenase obtained by inserting the DNA fragment containing the gene sequence to the vector to obtain the gene recombinant plasmid and inducing the gene recombinant plasmid with the inducer are relatively high, the crude enzyme mixed solution obtained after breaking the host strain and performing the centrifugation not only contains the phenylalanine dehydrogenase, but also contains the formate dehydrogenase contained in the nutritional methyl host strain itself, the present disclosure can catalyze conversion of a keto acid into an amino acid by using the crude enzyme mixed solution directly.

During an implementation process of the embodiment, the specific enzyme activities of both the phenylalanine dehydrogenase and the formate dehydrogenase in the crude enzyme mixed solution may be influenced by changes of the temperature and the culture medium, all crude enzyme mixed solutions obtained may be applied to the present disclosure, and in a preferred crude enzyme mixed solution obtained, the enzyme specific activity of the phenylalanine dehydrogenase is 40 U/ml to 60 U/ml and the enzyme specific activity of the formate dehydrogenase is 20 U/ml to 30 U/ml. The crude enzyme mixed solution having the enzyme specific activities has higher selectivity and catalytic efficiency in a conversion process of the keto acid into the L-heterocyclic amino acid.

In another preferred embodiment of the present disclosure, the Step B in the synthesis method comprises: adding the heterocyclic keto acid and ammonium formate to an aqueous solution, regulating the pH value to 8.2 to 8.5, adding the crude enzyme mixed solution and the coenzyme NAD⁺, and react at 30° C. to 40° C. until conversion of the raw materials is finished to obtain the L-heterocyclic amino acid. In the Step B, the water is used as a solvent, thus greatly reducing production costs and avoiding production of an organic solvent. The synthesis process is green and environment-friendly, which is further applicable to mass industrial production.

In order to further control costs by raw materials and regulate and control the proportion of each raw material to produce a product as much as possible, preferably, 2 ml to 10 ml of the crude enzyme mixed solution is added to each gram of the heterocyclic keto acid; 0.005 mole to 0.1 mole of the coenzyme NAD⁺ is added to each mole of the heterocyclic keto acid and 1.5 moles to 5 moles of ammonium formate is added to each mole of the heterocyclic keto acid.

In another preferred embodiment of the present disclosure, after the Step B, the synthesis method further comprises: adding concentrated hydrochloric acid to the system after the reaction, passing the system with the concentrated hydrochloric acid through diatomite to obtain a filtrate; regulating the pH value of the filtrate to 5.0 to 7.0, then passing the filtrate through a strong acid cation exchange resin to obtain a crude product; concentrating the crude product, regulating the pH value of the crude product to 7.0, adding an alcoholic solvent to wash the crude product and drying the washed crude product to obtain a purified L-heterocyclic amino acid. Since the present disclosure has higher chiral selectivity, and does not need to separate and purify the isomer from the obtained product, thus the separation method of the present disclosure is simple, and it only needs to separate the product from the enzymes and raw materials etc.

In another preferred embodiment of the present disclosure, a method for preparing the heterocyclic keto acid applied in the synthesis method comprises the following steps: subjecting a heterocyclic ketone with an acetic anhydride, a sodium acetate and an N-acetylglycine to reaction to obtain an intermediate product, wherein the heterocycle in the heterocyclic ketone is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, the structural formula of the ketone group in the heterocyclic ketone is —C═O and is located on any one of the carbon positions of the heterocyclic ketone; subjecting the intermediate product to a hydrolysis reaction in the presence of a Lewis base, and acidizing to obtain the heterocyclic keto acid.

The synthesis route of the heterocyclic keto acid is relatively short without a noble metal catalyst, thereby ensuring that there are no heavy metal residues in the obtained heterocyclic keto acid; use of a noble metal catalyst is also avoided in the subsequent synthesis process of the L-heterocyclic keto acid, thereby further ensuring that there are no heavy metal residues in the obtained L-heterocyclic keto acid.

In another preferred embodiment of the present disclosure, a method for preparing the heterocyclic keto acid applied in the synthesis method comprises the following steps: subjecting a heterocyclic alkyl compound with a diethyl oxalate in the presence of an N-butyllithium or a potassium tert-butoxide reaction to to generate a heterocyclic keto ester, wherein the heterocycle in the heterocyclic alkyl compound is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, the alkyl in the heterocyclic alkyl compound is methyl and is located on any one of the carbon positions of the heterocyclic alkyl compound; subjecting the heterocyclic keto ester to a hydrolysis reaction in the presence of a Lewis base, and acidizing to obtain the heterocyclic keto acid.

Similarly, the synthesis route of the heterocyclic keto acid is relatively short without a noble metal catalyst, thereby ensuring that there are no heavy metal residues in the obtained heterocyclic keto acid; use of a noble metal catalyst is also avoided in the subsequent synthesis process of the L-heterocyclic keto acid, thereby further ensuring that there are no heavy metal residues in the obtained L-heterocyclic keto acid.

A pharmaceutical composition is provided in another typical embodiment of the present disclosure, the pharmaceutical composition comprises an effective dose of a L-heterocyclic keto acid and a pharmaceutical vector, the L-heterocyclic keto acid synthesized and obtained by the synthesis method above. The L-heterocyclic amino acid of the present disclosure is relatively high in purity, therefore the pharmaceutical composition having the L-heterocyclic amino has a smaller target and lower side effects compared with a pharmaceutical composition having an L-heterocyclic amino acid in the prior art.

The beneficial effect of the present disclosure will be further described hereinafter in combination with embodiments and comparison examples.

The phenylalanine dehydrogenase used in the following embodiments is a phenylalanine dehydrogenase having an amino acid sequence of SEQ ID No. 1, wherein a gene sequence coding the phenylalanine dehydrogenase of the 1^{st} embodiment to the 8^{th} embodiment is from bacillus sphaericus

An expression process of the phenylalanine dehydrogenase is as follows:

DNA fragments containing the gene sequence of SEQ ID No. 2 were inserted to a pET-22b(+) vector to obtain gene recombinant plasmids, the gene recombinant plasmids were transferred to escherichia colis BL21, the escherichia colis BL21 were cultured on a culture medium, the phenylalanine dehydrogenases were induced production by an inducer, the escherichia colis BL21 were broken with ultrasonic waves, and then centrifugal separation was carried out to obtain a crude enzyme mixed solution having the phenylalanine dehydrogenase with a specific enzyme activity of 38 to 70 U/ml and a formate dehydrogenase with a specific enzyme activity of 15 to 35 U/ml.

### Embodiment 1

### Synthesis of L-4-pyridylalanine

Step 1: 904.2 g of 1.5eq potassium tert-butoxide, 2 L of tetrahydrofuran and 500 g of 4-methylpyridine were added to a 4-neck flask, stirred for 2.5 h at room temperature, 941.1 g of diethyl oxalate was dropwise added, after finishing the addition, stir was performed overnight at room temperature until the reaction was finished, wherein the specific reaction is expressed by the following formula. The system was temporarily stored and be used directly in the second step.

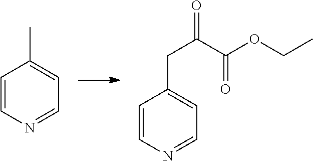

Step 2: the previous system was added to a bottle, 1 L of methanol, 2 L of H2O, 783.6 g of potassium tert-butoxide were added to the bottle, react was performed while preserving the temperature until there was no raw material, wherein the specific reaction is expressed by the following formula. Then concentration was performed, the concentrated system was cooled to room temperature, the pH value was regulated to 2 to 3 with hydrochloric acid having a concentration of 6 mol/L, water was added having a volume which is 3 times as large as the system with pH value of 2 to 3 to dilute the system, suction filtration was performed, an obtained filtrate was cooled to 0 to 5° C. and then performed suction filtration to obtain 640 g of a solid, the yield of these two steps is 72.2%. 1H NMR (400 MHz, DMSO): δ 8.45 (d, 2H), 8.03 (d, 2H), 7.55 (d, 1H).

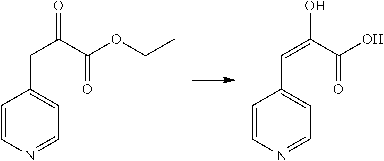

Step 3: 150 ml of purified water and 12.1 g of sodium hydroxide were added to a 2 L four-neck flask and stirred until they are fully dissolved, then 50 g of keto acid was added and stirred until the whole system was fully dissolved, the pH value was detected to be 9 to 10, 28.6 g of ammonium formate was added, the pH value of the system was regulated to 8.2 to 8.5 with NaOH, 500 mL of a crude enzyme mixed solution having a phenylalanine dehydrogenase with a specific enzyme activity of 50 U/ml and a formate dehydrogenase having a specific enzyme activity of 25 U/ml and 2.0 g of NAD+ was added, then the temperature of the system was increased to 30 to 40° C. and reacted was performed until there was no raw material, wherein the specific equation is as follows. The pH value of the reacted system was regulated to 1 to 2 with 100 mL of concentrated hydrochloric acid, and passed through diatomite to obtain a filtrate, the pH value of the filtrate was regulated to 6 to 7 with NaOH, and passed the filtrate through a strong acid salt exchange resin to obtain a crude product; the product was concentrated and then the pH value of the crude product was regulated to 7 with formic acid, and ethanol was added to wash the crude product to obtain an almost white solid, an L-heterocyclic amino acid having a chiral purity of 99.5%. 1H NMR (400 MHz, D2O): δ8.58 (d, 2H), 7.90 (d, 2H), 4.45 (t, 1H), 3.47 (dd, 2H).

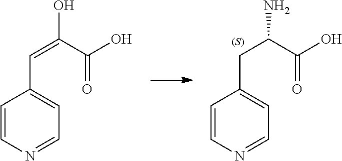

### Embodiment 2

### Synthesis of L-2-pyridylalanine

Step 1: 800 L of tetrahydrofuran, and 152 g of (1.4eg) redistilled diisopropylamine were added to a 2 L four-neck flask, then stirred and cooled to −50° C. to −40° C., 590 mL of n-butyllithium (2.55N, 1.4eq) was dropwise added at −50 to −40° C., then stirred for 0.5 h and cooled to −80 to −70° C., 100 g of 2-methylpyridine at −80 to −70° C. was dropwise added, then reaction was performed for 2 h while preserving the temperature, and then Thin Layer Chromatography (TLC) was performed to tracked until there was no raw material to obtain system A.

200 mL of tetrahydrofuran and 172 g of diethyl oxalate were added to a 3 L four-neck flask, stirred uniformly and then cooled to −80 to −70° C. to obtain system B.

System A was pressed into system B at −80 to −70° C., stirred for 1 h, then TLC was performed to tracked until the reaction was finished, the temperature was increased to −60° C., the system temperature was controlled to below −20° C., the pH of the system was regulated to 5 to 6 with hydrochloric acid having a concentration of 2 mol/L, the system temperature was increased to room temperature, liquid separation was performed and then a water phase was extracted with 300 mL of ethyl acetate for three times respectively, organic phases obtained after the extraction were combined and dried overnight with sulfuric acid, the dried organic phase was performed suction filtration, and the mother liquor was concentrated until a great amount of solids were precipitated, and suction filtration was performed continually to obtain 105 g of a crude product 1.

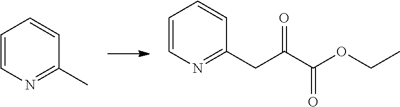

Step 2: 100 g of the crude product 1 was dissolved in 200 mL of a sodium hydroxide solution having a concentration of 2 mol/L, the temperature was increased to 60 to 70° C., reaction was performed for 6 to 8 h while preserving the temperature, TLC was performed to tracked until the reaction was finished, the temperature was cooled to 15 to 25° C. and then the system was washed with 200 mL of ethyl acetate, an obtained water phase was cooled to 0 to 5° C., and the pH value of the water phase was regulated to 1 to 2 at 0 to 5° C. with concentrated hydrochloric acid to precipitate a solid, suction filtration was performed, and a filter cake was washed with 40 mL of ice water to obtain 26 g of a crude product 2. 1H NMR (400 MHz, DMSO): δ8.50 (d, 1H), 7.89 (t, 1H), 7.47 (d, 1H), 7.29 (t, 1H), 6.53 (s, 1H).

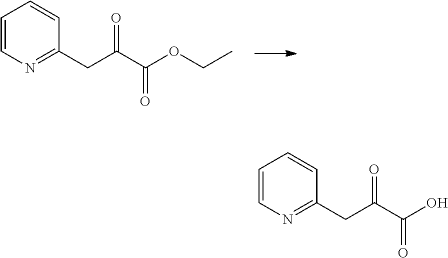

Step 3: 7.91 g of NaOH, 100 mL of purified water, 32.6 g of keto acid, and 24.9 g of ammonium formate were added to a 1 L four-neck flask, wherein the pH value of the system was 8.2 to 8.5, 326 ml of a crude enzyme mixed solution having a phenylalanine dehydrogenase having a specific enzyme activity of 40 U/ml and a formate dehydrogenase having a specific enzyme activity of 30 U/ml and 1.31 g of NAD+ were added to the 1 L four-neck flask, then the temperature of the system was increased to 30 to 40° C., reaction was performed overnight and then tracked until conversion of the raw materials was finished, then 65 mL of concentrated hydrochloric acid was added to the system, the system was passed through diatomite to obtain a filtrate, the pH value of the filtrate was regulated to 7 with NaOH, then the filtrate was passed through a strong acid cation exchange resin to obtain a crude product 3, the crude product 3 was concentrated, and then the pH value of the crude product was regulated 3 to 7 with formic acid, and the crude product 3 was washed with isopropanol to obtain 12.8 g of a solid, an L-heterocyclic amino acid having a chiral purity of 99.6%. 1H NMR (400 MHz, D2O): δ8.66 (d, 1H), 8.48 (t, 1H), 7.97 (d, 1H), 7.91 (t, 1H), 4.23 (t, 1H), 3.58 (d, 2H).

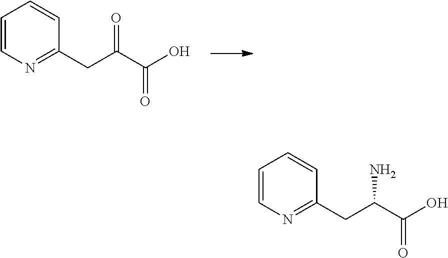 [SMILES: O=C(O)C(=O)Cc1ccccn1 ; N[C@@H](Cc1ccccn1)C(=O)O]

### Embodiment 3

### Synthesis of L-3-pyridylalanine

Step 1: 5.7 L of acetic anhydride, 946 g of sodium acetate, 1350 g of acetylglycine and 950 g of 3-pyridinecarboxaldehyde were added to a 20 L four-neck flask, the temperature was increased to 100 to 105° C. and then reacted for 4 h, the system temperature was cooled to below 5° C., suction filtration was performed, and a filter cake was washed with ice water to obtain 1100 g of a solid product 1. ¹H NMR (400 MHz, CDCl₃): δ9.01 (s, 1H), 8.58 (d, 1H), 8.55 (d, 1H), 7.38 (t, 1H), 7.04 (s, 1H), 2.15 (s, 3H);

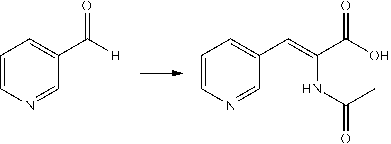

Step 2: 1100 g of the solid product 1, 5.5 L of dioxane and 5.5 L of hydrochloric acid having a concentration of 4 mol/L were added to a 20 L four-neck flask, the system was subjected to a reflux reaction for 3.5 h and then cooled to room temperature, then the system was concentrated until most liquid was steamed, then the concentrated system was subjected to suction filtration, a filter cake was washed with 550 mL of ice water, and then washed with 550 mL of acetone to obtain 391 g of a yellow solid 2. 1H NMR (400 MHz, DMSO): δ10.13 (d, 1H), 9.80 (d, 1H), 9.72 (d, 1H), 9.01 (t, 1H), 7.56 (s, 1H).

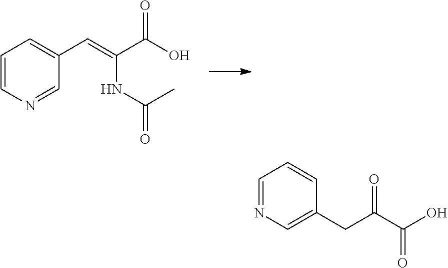

Step 3: pure water and 36 g of NaOH were added to a 3 L four-neck flask at room temperature, 60 g of 2-carbonyl-3-(pyridine-3-yl)propionic acid was added to the system, stirred until full dissolution was dissolved, 45.4 g of ammonium formate, 600 mL of a crude enzyme mixed solution having a phenylalanine dehydrogenase having a specific enzyme activity of 60 U/ml and a formate dehydrogenase having a specific enzyme activity of 20 U/ml and 2.39 g of β-NAD+ were added to the system, the pH value of the system was regulated to 8.5 with concentrated ammonia, and then the temperature was increased to 30 to 40° C. and reacted for 4 days, 4 days later, 100 mL of concentrated hydrochloric acid was slowly added in a dropwise manner to the system to regulate the pH value of the system to 1 to 2, liquid separation was performed, then a water phase was passed through 1 to 2 cm diatomite to obtain a filtrate, the pH value of the filtrate was regulated to 1 to 2 with sodium hydroxide in an ice bath, filtrates was blended having a feed amount of 63 g and 50 g and obtained through the process above, and passed through a resin column to be purified (the type of the resin column is a 001×7 strong acid cation exchange resin with a resin amount of 15 L), 90 mL and 60 mL of pure water were added in turn to an obtained crude product to wash the crude product while stirring in an ice salt bath, suction filtration was performed to obtained a filter cake, and wash the filter cake with 100 mL of isopropanol and 80 mL of absolute ethyl alcohol respectively and dried to obtain 77 g of a light yellow solid, an L-heterocyclic amino acid having a chiral purity of 99.5%. 1H NMR (400 MHz, D2O): δ8.43 (d, 2H), 7.87 (d, 1H), 7.50 (t, 1H), 3.97 (t, 1H), 3.24 (dd, 2H).

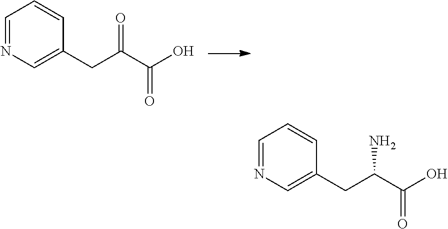 [SMILES: O=C(O)C(=O)Cc1cccnc1 ; N[C@@H](Cc1cccnc1)C(=O)O]

### Embodiment 4

### Synthesis of L-2-pyrazolylalanine

Step 1: protected by nitrogen having a temperature of −20° C., 244 ml of n-butyllithium was dropwise added to 560 ml of a tetrahydrofuran solution in which 50 g of compound 1 was dissolved, the mixture was stirred at −20° C. for 1 h and then 135 g of dimethylformamide was added to the mixture gradually in a dropwise manner, stir was performed continually for 2 h, wherein the specific equation is as follows, the reactants was quenched with ammonium chloride having a concentration of 1 mol/L, concentration was performed to remove tetrahydrofuran, and the residues was dissolved in ethylamine and water, separated, washed and combined organic layer was washed with brine and dried with Na₂SO₄, and the organic layer was filtered and concentrated to obtain a brown oily crude product 2 which is used in the next step directly.

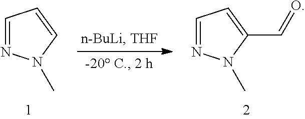

Step 2: 17.4 g of compound E and 63 g of compound 2 were added to 286 ml of an aqueous solution containing 57.2 g of compound D at room temperature to form a mixture, the mixture was heated to 100° C. The mixture was stirred for 3 h and then cooled to room temperature, and the mixture was filtered to obtain a precipitate, the precipitate was washed with water and then dried to obtain 45 g of a yellow solid, i.e. compound 3. 1H NMR (400 MHz, DMSO): δ7.47 (d, 1H), 6.93 (d, 1H), 6.34 (s, 1H), 3.88 (s, 3H).

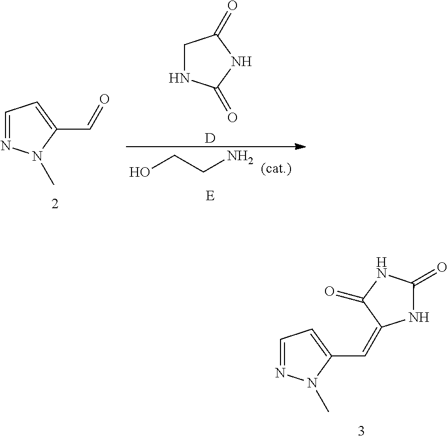

Step 3: 50 g of compound 3 was added to 300 ml of an aqueous solution in which 52 g of NaOH is dissolved to obtain a mixed solution, the obtained mixed solution was heated to 100° C., and stirred for 2 h, then cooled to room temperature to obtain a mixture, wherein the specific equation is as follows, the pH value of the mixture was regulated to 3 to 4 with concentrated hydrochloric acid and filtered to obtain a precipitate, the precipitate was washed with water and dried to obtain 26 g of a white solid product with a yield of 59%. 1H NMR (400 MHz, DMSO): δ7.68 (d, 1H), 6.96 (d, 1H), 6.68 (s, 1H), 4.09 (s, 3H).

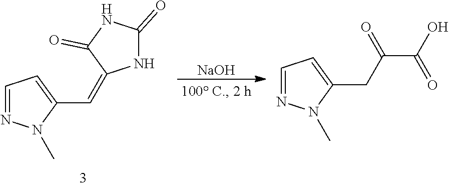

Step 4: 2.0 g of the white solid product obtained in the previous step and 1.51 g of ammonium formate were added to 10 mL of an aqueous solution in which 0.475 g of NaOH is dissolved and obtained mixture with the pH value of 7.5 to 8.0, 20 mL of a crude enzyme mixed solution having a phenylalanine dehydrogenase having a specific enzyme activity of 50 U/ml and a formate dehydrogenase having a specific enzyme activity of 25 U/ml and 80 mg of NAD+ were added to the mixture, the pH value of the mixture was regulated to 8.5 with ammonia, and then the mixture was subjected to react at 30° C. for 7 days, wherein the specific equation is as follows, 7 days layer, about 2.5 ml of concentrated hydrochloric acid was added to the mixture, the mixture with the concentrated hydrochloric acid was passed through diatomite to filter, the pH value of a filtrate was regulated to 7.0 with NaOH, and passed through a strong acid cation exchange resin to obtain a purified product 1, an L-heterocyclic amino acid having a chiral purity of 98.5%. 1H NMR (400 MHz, D2O): δ8.50 (d, 2H), 3.5 (t, 1H), 2.87 (d, 2H).

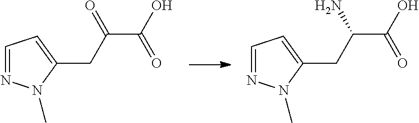

### Embodiment 5

### Synthesis of L-2-thienylalanine

Step 1: 196.6 g of 2-formylthiophene, 267 g of N-acetylglycine, 187 g of sodium acetate and 1180 mL of acetic anhydride were added to a 2 L four-neck flask, the temperature of the system was increased to 100 to 110° C., and then reaction was performed for about 24 h, track was performed until conversion of the raw materials were finished, wherein the specific equation is as follows, the system was cooled to room temperature, 540 mL of n-heptane was added to the system and then suction filtration was performed, an obtained filter cake was washed with 2 L of ice water, and dried to obtain 154.8 g of a crude product with a yield of 45.7%. ¹H NMR (400 MHz, CDCl3): δ7.60 (d, 1H), 7.48 (d, 1H), 7.03 (t, 1H), 2.28 (s, 3H).

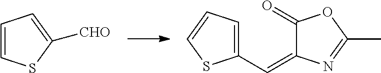

Step 2: 81.84 g of the crude product, 654 mL of water, and 266.6 g of LiOH—H2O was added to a 1 L four-neck flask, stirred and the temperature of the system was increased to 60 to 70° C. and reacted for about 11 h, and track was performed until conversion of the raw materials is finished, wherein the specific equation is as follows, the temperature of the reacted system was cooled to room temperature and 580 mL of concentrated hydrochloric acid was dropwise added to it to regulate the pH value to 3, suction filtration was performed to precipitate a solid, the solid was washed and then used in the reaction of the next step directly. 1H NMR (400 MHz, DMSO): δ7.39 (d, 1H), 7.09 (d, 1H), 6.99 (t, 1H), 6.50 (s, 1H).

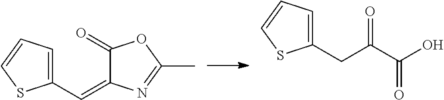

Step 3: 11.7 g of NaOH and 250 mL of pure water were added to a 2 L four-neck flask, after they were fully dissolved, 50 g of keto acid and 36.9 g of ammonium formate were added, the pH value of the formed system was regulated to 8.2 to 8.5, then 500 mL of a crude enzyme mixed solution having a phenylalanine dehydrogenase having a specific enzyme activity of 50 U/ml and a formate dehydrogenase having a specific enzyme activity of 25 U/ml and 1.94 g of NAD+ were added to the system, the temperature of the system was increased to 30° C. to 40° C., and then subjected to react for about 40 h, and track performed until reaction of the raw materials was finished, 100 mL of concentrated hydrochloric acid was dropwise added to the system continually to terminate the reaction, the system was passed through diatomite to obtain a filtrate, the pH value of the obtained filtrate was regulated to 5 to 6 with a sodium hydroxide solid, and continue to cooled to −18° C. to crystallize, the mother liquor was passed through a strong acid cation exchange resin to recycle products, combined all products were combined and washed with isopropanol and dried to obtain an almost white solid, an L-heterocyclic amino acid having a chiral purity of 99.7%. 1H NMR (400 MHz, D2O): δ7.30 (d, 1H), 7.02 (t, 1H), 6.93 (s, 1H), 3.50 (t, 1H, 3.15 (d, 2H).

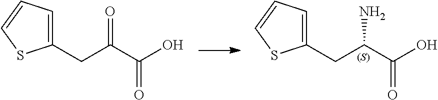

### Embodiment 6

### Synthesis of L-3-thienylalanine

Step 1: 25 g of 3-formylthiophene, 33.9 g of N-acetylglycine, 150 mL of acetic anhydride and 23.8 g of sodium acetate were added to a 500 mL four-neck flask, stirred and the temperature was increased to 97° C. to 103° C., then reacted for 2 h, wherein the specific equation is as follows, the system was cooled to room temperature after the reaction, and poured into 200 g of ice water, suction filtration was performed, and a filter cake was washed with 100 mL of water to obtain 28.7 g of a yellow solid product 1.

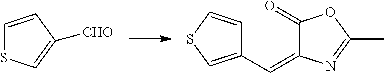

Step 2: 23.8 g of the yellow solid 1, 77.6 g of lithium hydroxide monohydrate and 190 mL of water were added to a 1 L four-neck flask, the temperature was increased to 50 to 60° C., and reaction was subjected for 2 h, and then 50 mL of methanol was added, the reaction was subjected continually for about 3 h and then the system was cooled to room temperature, the temperature of the system was controlled to below 20° C., and the pH of the system was regulated to 1 to 2 with hydrochloric acid having a concentration of 6 mol/L, then 600 mL of ethylamine was added to the system and the system with the ethylamine was filtered, and then an obtained water phase was extracted twice with 300 mL of ethylamine, the organic phases obtained from the extraction were combined and then decolorized with activated carbon to obtain a crude product, the crude product was concentrated and washed with 40 mL of dichloromethane to obtain 5.75 g of a product 2. 1H NMR (400 MHz, (CD2)2CO) 7.83 (d, 1H), 7.51 (d, 1H), 7.46 (d, 1H), 6.67 (s, 1H).

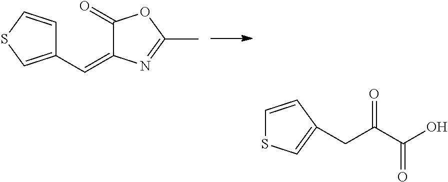

Step 3: 1.0 g of keto acid, 2 mL of pure water, 1.11 g of ammonium formate, 39 mg of NAD+, and 11.3 mL of a crude enzyme mixed solution having a phenylalanine dehydrogenase having a specific enzyme activity of 60 U/ml and a formate dehydrogenase having a specific enzyme activity of 20 U/ml were added to a 50 mL reaction bottle to obtain a system with the pH value of 8.2 to 8.5, the system reacted at 30° C. to 40° C., about 3 days later, tracking was performed until conversion of the raw materials were finished, 6 mL of concentrated hydrochloric acid was added to the system to terminate the reaction, the system was passed through diatomite to obtain a filtrate, the pH value of the filtrate was regulated with NaOH was regulated to 5 to 6, then the filtrate was purified with a strong acid cation exchange resin to obtain 0.2 g of a target compound, an L-heterocyclic amino acid having a chiral purity of 99.5%. 1H NMR (400 MHz, D2O): δ7.51 (d, 1H), 7.26 (s, 1H), 7.11 (d, 1H), 3.58 (t, 1H), 3.04 (d, 2H).

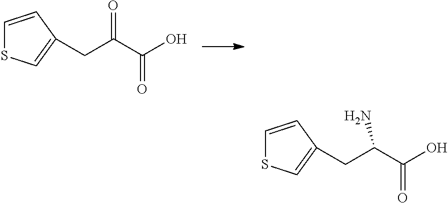 [SMILES: O=C(O)C(=O)Cc1ccsc1 ; N[C@@H](Cc1ccsc1)C(=O)O]

### Embodiment 7

### Synthesis of L-4-pyridylalanine

Step 1: 904.2 g of 1.5eq potassium tert-butoxide, 2 L of tetrahydrofuran and 500 g of 4-methylpyridine were added to a four-neck flash, and stirred for 2.5 h at room temperature, 941.1 g of diethyl oxalate was dropwise added, after the addition was finished, stirred overnight at room temperature until the reaction was finished, wherein the specific reaction is expressed by the following formula. The system was stored temporarily and used in the next step directly;

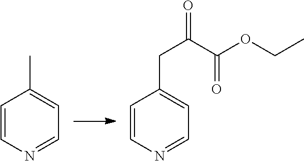

Step 2: the previous system was added to a bottle, 1 L of methanol, 2 L of H2O, and 783.6 g of potassium tert-butoxide were added to the bottle, reaction was subjected while preserving the temperature until there is no raw material, wherein the specific reaction is expressed by the following formula. Then the system was concentrated and cooled to room temperature, the pH value of the system with room temperature was regulated to 2 to 3 with hydrochloric acid having a concentration of 6 mol/L, water having a volume that is three times as large as that of the system was added to dilute the system, suction filtration was performed to obtain a filtrate, the filtrate was cooled to 0 to 5° C., and the cooled filtrate was subjected suction filtration to obtain 640 g of a solid, wherein the yield of these two steps is 72.2%. 1H NMR (400 MHz, DMSO): δ8.45 (d, 2H), 8.03 (d, 2H), 7.55 (d, 1H).

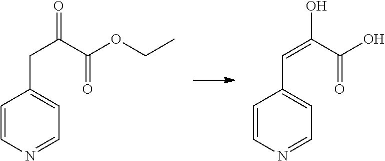

Step 3) Condition 1:

150 mL of purified water and 12.1 g of sodium hydroxide was added to a 2 L four-neck flask, stirred until fully dissolved, then 50 g of keto acid was added, stirred continually until the whole system were fully dissolved, and the pH value of the system was 9 to 10, 28.6 g of ammonium formate was added to the system, the pH value of the system with ammonium formate was regulated to 8.2 to 8.5 with NaOH, 500 ml of a crude enzyme mixed solution having a phenylalanine dehydrogenase having a specific enzyme activity of 70 U/ml and a formate dehydrogenase having a specific enzyme activity of 35 U/ml, and 2.0 g of NAD⁺ were added to the system, the temperature of the system was increased to 30° C. to 40° C., and the system was subjected to react until there was no raw material, wherein the specific equation is as follows. The pH value of the system after the reaction was regulated to 1 to 2 with 100 mL of concentrated hydrochloric acid, and passed through diatomite to obtain a filtrate, then the pH value of the filtrate was regulated to 6 to 7 with NaOH and passed through a strong acid cation exchange resin to obtain a crude product; the crude product was concentrated and then the pH value thereof was regulated to 7.0 with formic acid, and the crude product with the pH value of 7.0 was washed with ethanol to obtain an almost white solid having a chiral purity of 99.1%. ¹H NMR (400 MHz, D2O): δ8.58 (d, 2H), 7.90 (d, 2H), 4.45 (t, 1H), 3.47 (dd, 2H).

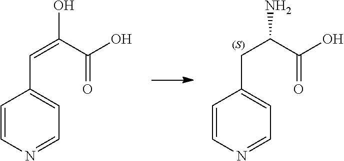

Condition 2:

150 mL of purified water and 12.1 g of sodium hydroxide were added to a 2 L four-neck flask, stirred until dissolved, then 50 g of keto acid was added, stirred continually until the whole system was fully dissolved and the pH value was 9 to 10, 28.6 g of ammonium formate was add to the system above, the pH value of the system was regulated to 7.5 to 8.0 with NaOH, 500 mL of a crude enzyme mixed solution having a phenylalanine dehydrogenase having a specific enzyme activity of 70 U/ml and a formate dehydrogenase having a specific enzyme activity of 35 U/ml, and 2.0 g of NAD+ were added to the system, the temperature of the system was increased to 25 to 27° C., and the system was subjected to react until there was no raw material, wherein the specific equation is as follows. The pH value of the system after the reaction was regulated to 1 to 2 with 100 mL of concentrated hydrochloric acid and passed through diatomite to obtain a filtrate, the pH value of the filtrate was regulated to about 4.5 with NaOH, then the filtrate with the pH value of 4.5 was passed through a strong acid cation exchange resin to obtain a crude product; the crude product was concentrate and then regulated to a pH value of 8.0 with formic acid, and the crude product was washed with ethanol to obtain an almost white solid having a chiral purity of 99.1%. ¹H NMR (400 MHz, D₂O): δ8.58 (d, 2H), 7.90 (d, 2H), 4.45 (t, 1H), 3.47 (dd, 2H).

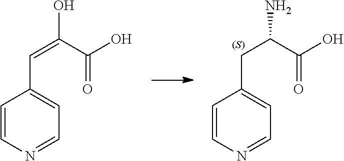

### Comparison Example 1

L-methyl-2-acetamino-3-(4-pyridyl)-methyl propionate was synthesized according to a method recorded in literature “Transition-Metal-Assisted Asymmetric Synthesis of Amino Acid Analogues. A New Synthesis of Optically Pure D- and L-Pyridylalanines”.

500 mg of methyl-2-acetamide-3-(4-pyridyl)vinyl formate, 60 mg of (R,R)Rh(DIPAMP)(COD)HBF⁴⁻) and 12 mL of methanol were added to a high pressure kettle, H₂ was introduced to subject the pressure reach 65 psi, tracking was performed until the reaction was finished, then column chromatographic separation was performed to obtain 360 mg of a product, and a solid intermediate was precipitated after placing the product for 3 months, verified structural data is as follows: ¹H NMR (CDCl3) 1.99 (s, 3H, NHC—(OICH), 3.07 (dd, 1H, CH₂CH), 3.18 (dd, 1H, CH₂CH), 3.75 (8.3 H, COOCH₃), 4.94 (9, 1 H, CH₂CH), 6.88 (br s, 1H, NH), 7.08 (d, 2H, aromatic), 8.45 (d, 2H, aromatic); ‘3c NMR (CDCld 22.51, 36.81, 52.17, 123.11, 124.31, 145.42, 149.31, 169.88, 171.4; IR (neat) 3272, 3038, 2955, 1744, 1661, 1605, 1549, 1437, 1420, 1374, 1285, 1217, 1179, 1003; MS (CI) [M+HI m/e 223]; GC, 97:3, the chiral purity of the intermediate is 94%, and the intermediate was subjected to hydrolysis with hydrochloric acid having a concentration of 6 mol/L to obtain an L-amino acid having a chiral purity of 96%.

### Comparison Example 2

A reaction of converting a pyridine keto acid into an amino acid was catalyzed by an L-leucine dehydrogenase and a formate dehydrogenase, and no product was detected by Nuclear Magnetic Resonance (NMR) monitor.

Chiral purities of L-heterocyclic amino acids of the 1^{st} embodiment to the 7^{th} embodiment and the 1^{st} comparison example to the 2^{nd} comparison example were obtained by an NMR internal standard and recorded in Table 1.

TABLE 1

| | Embodi- | Embodi- | Embodi- | Embodi- | Embodi- |
|---|---|---|---|---|---|
| | ment 1 | ment 2 | ment 3 | ment 4 | ment 5 |
| Chiral | 99.5 | 99.6 | 99.5 | 98.5 | 99.7 |
| purity | | | | | |
| (%) | | | | | |

| | Embodi- | Embodi- | Comparison | Comparison |
|---|---|---|---|---|
| | ment 6 | ment 7 | example 1 | example 2 |
| Chiral | 99.5 | 99.1 | 96 | — |
| purity | | | | |
| (%) | | | | |

It can be learned from the data in Table 1 that the chiral purities of the L-heterocyclic amino acids prepared in the 1st embodiment to the 7th embodiment by using the preparation method of the present disclosure are higher than 98%. In addition, it is founded by comparing the 1st embodiment with the 1st and 2nd embodiments that the chiral purity of an L-heterocyclic amino acid using the specific phenylalanine dehydrogenase of the present disclosure is improved obviously.

The above are only preferred embodiments of the present disclosure and should not be used for limiting the present disclosure. For those skilled in the art, the present disclosure may have various modifications and changes. Any modifications, equivalent replacements, improvements and the like within the spirit and principle of the present disclosure shall fall within the scope of protection of the present disclosure.

1. A synthesis method for L-heterocyclic amino acid, wherein the synthesis method comprises:
Step A: preparing a heterocyclic keto acid, wherein the heterocycle in the heterocyclic keto acid is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, and wherein the keto acid group in the heterocyclic keto acid has a structural formula of: 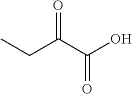
and is located on any one of the carbon positions of the heterocycle, and
Step B: mixing the heterocyclic keto acid with ammonium formate, a phenylalanine dehydrogenase, a formate dehydrogenase and a coenzyme NAD⁺, and carrying out a reductive amination reaction to generate the L-heterocyclic amino acid, wherein the amino acid sequence of the phenylalanine dehydrogenase is SEQ ID No. 1.

2. The synthesis method according to claim 1, wherein a gene sequence coding the phenylalanine dehydrogenase is SEQ ID No. 2.

3. The synthesis method according to claim 2, wherein an expression process of the phenylalanine dehydrogenase comprises:
inserting a DNA fragment containing the gene sequence into a vector to obtain a gene recombinant plasmid;
transferring the gene recombinant plasmid to a host strain and culturing the host strain on a culture medium, and inducing production of the phenylalanine dehydrogenase by an inducer;
breaking the host strain with ultrasonic waves, and then carrying out centrifugal separation to obtain a crude enzyme mixed solution which contains the phenylalanine dehydrogenase and the formate dehydrogenase.

4. The synthesis method according to claim 3, wherein in the crude enzyme mixed solution, the specific enzyme activity of the phenylalanine dehydrogenase is 40 U/ml to 60 U/ml, and the specific enzyme activity of the formate dehydrogenase is 20 U/ml to 30 U/ml.

5. The synthesis method according to claim 3, wherein the Step B comprises:
adding the heterocyclic keto acid and the ammonium formate to an aqueous solution, regulating the pH value to 8.2 to 8.5, adding the crude enzyme mixed solution and the coenzyme NAD⁺, and performing reaction at 30° C. to 40° C. until conversion of the raw materials is finished to obtain the L-heterocyclic amino acid.

6. The synthesis method according to claim 5, wherein
2 ml to 10 ml of the crude enzyme mixed solution is added to each mole of the heterocyclic keto acid;
0.005 mole to 0.1 mole of the coenzyme NAD⁺ is added to each mole of the heterocyclic keto acid and 1.5 moles to 5 moles of the ammonium formate is added to each mole of the heterocyclic keto acid.

7. The synthesis method according to claim 1, wherein after the Step B, the synthesis method further comprises:
adding concentrated hydrochloric acid to the system after the reaction, passing the system with the concentrated hydrochloric acid through diatomite to obtain a filtrate;
regulating the pH value of the filtrate to 5.0 to 7.0, then passing the filtrate through a strong acid cation exchange resin to obtain a crude product;
concentrating the crude product, adding an alcoholic solvent to wash the crude product and drying the washed crude product to obtain a purified L-heterocyclic amino acid.

8. The synthesis method according to claim 1, wherein a method for preparing the heterocyclic keto acid comprises the following steps:
subjecting a heterocyclic ketone with an acetic anhydride, a sodium acetate and an N-acetylglycine to reaction to obtain an intermediate product, wherein the heterocycle in the heterocyclic ketone is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle; the structural formula of the ketone group in the heterocyclic ketone is —C═O and is located on any one of the carbon positions of the heterocyclic ketone;
subjecting the intermediate product to a hydrolysis reaction in the presence of a Lewis base, and acidizing to obtain the heterocyclic keto acid.

9. The synthesis method according to claim 1, wherein a method for preparing the heterocyclic keto acid comprises the following steps:
subjecting a heterocyclic alkyl compound with a diethyl oxalate in the presence of an N-butyllithium or a potassium tert-butoxide to reaction to generate a heterocyclic keto ester, wherein the heterocycle in the heterocyclic alkyl compound is selected from any one of a five-membered heterocycle, a six-membered heterocycle, a seven-membered heterocycle, an alkyl-substituted five-membered heterocycle, an alkyl-substituted six-membered heterocycle, and an alkyl-substituted seven-membered heterocycle, the alkyl in the heterocyclic alkyl compound is methyl and is located on any one of the carbon positions of the heterocyclic alkyl compound;
subjecting the heterocyclic keto ester to a hydrolysis reaction in the presence of a Lewis base, acidizing to obtain the heterocyclic keto acid.

10. A pharmaceutical composition, wherein it comprises an effective dose of a L-heterocyclic keto acid and a pharmaceutical vector, the L-heterocyclic keto acid is synthesized and obtained by the synthesis method according claim 1.

11. The synthesis method according to claim 4, wherein the Step B comprises:
adding the heterocyclic keto acid and the ammonium formate to an aqueous solution, regulating the pH value to 8.2 to 8.5, adding the crude enzyme mixed solution and the coenzyme NAD+, and performing reaction at 30° C. to 40° C. until conversion of the raw materials is finished to obtain the L-heterocyclic amino acid.

8. The synthesis method according to claim 6, wherein:
2 ml to 10 ml of the crude enzyme mixed solution is added to each mole of the heterocyclic keto acid; and
0.005 mole to 0.1 mole of the coenzyme NAD+ is added to each mole of the heterocyclic keto acid and 1.5 moles to 5 moles of the ammonium formate is added to each mole of the heterocyclic keto acid.
